(19) United States
(12) Patent Application Publication
(10) Pub. No.: US 2007/0135814 A1
(43) Pub. Date: Jun. 14, 2007

(54) Facet spacer

(75) Inventor: Robert A. Farris, Cordova, TN (US)

Correspondence Address: COATS & BENNETT, PLLC, 1400 Crescent Green, Suite 300, Cary, NC, 27518, US

(73) Assignee: SDGI Holdings, Inc.

(21) Appl. No.: 11/301,048

(22) Filed: Dec. 12, 2005

(51) Int. Cl. A61B 17/70; A61F 2/44

(52) U.S. Cl. 606/61; 623/17.13

(57) ABSTRACT

Devices for insertion within a facet joint formed between adjacent vertebral members. The devices include an intermediate section sized to fit within the facet joint. The devices may also include one or more mounting sections that extend across the face of one or both vertebral members to receive a fastener for mounting the device. Methods of using the devices may include positioning the intermediate section within the facet joint and connecting the device to one or both vertebral members.

### BACKGROUND

The present application is directed to vertebral spacers, and more specifically, to spacers for use with a facet joint.

The vertebral column includes thirty-three vertebrae. Each vertebrae includes an anterior body and a posterior arch. The posterior arch includes two pedicles and two laminae that join together to form the spinous process. A transverse process is laterally positioned at the transition from the pedicles to the laminae. Both the spinous process and transverse process provide for attachment of muscle. Two inferior articular processes extend downward from the junction of the laminae and the transverse process. Further, two superior articular processes extend upward from the junction.

Facet joints are formed by the articular processes of adjacent vertebrae. The inferior articular process of one vertebra articulates with the superior articular process of the vertebra below. The facet joints are synovial gliding joints because the articular surfaces glide over each other. They are capsular joints containing synovial fluid for lubrication. The facet joints are oriented in different planes depending upon their anatomic location. The orientation of the facets control the type and amount of joint motion.

### SUMMARY

The present application is directed to a device inserted within a facet joint formed between adjacent vertebral members. The device includes an intermediate section sized to fit within the facet joint. The device may also include a mounting section that extends across the face of one or both vertebral members to receive a fastener for mounting the device.

### BRIEF DESCRIPTION OF THE DRAWINGS

FIG. 1 is a perspective view of a spacer positioned within the facet joint according to one embodiment;

FIG. 2 is an exploded perspective view of a spacer and mounting screws according to one embodiment;

FIG. 3 is a side schematic view of a spacer according to one embodiment;

FIG. 4 is a rear view of a pair of spacers mounted within facet joints of adjacent vertebral members according to one embodiment;

FIGS. 5A and 5B are perspective views of inserting the spacer into the patient according to one embodiment;

FIG. 6 is an exploded perspective view of a spacer, mounting screws, and member according to one embodiment;

FIG. 7 is a perspective view of a spacer and mounting screw according to one embodiment;

FIG. 8 is a perspective view of a spacer positioned within a facet joint according to one embodiment;

FIG. 9 is a perspective view of a spacer and mounting screw according to one embodiment; and

FIG. 10 is a perspective view of a spacer according to one embodiment.

### DETAILED DESCRIPTION

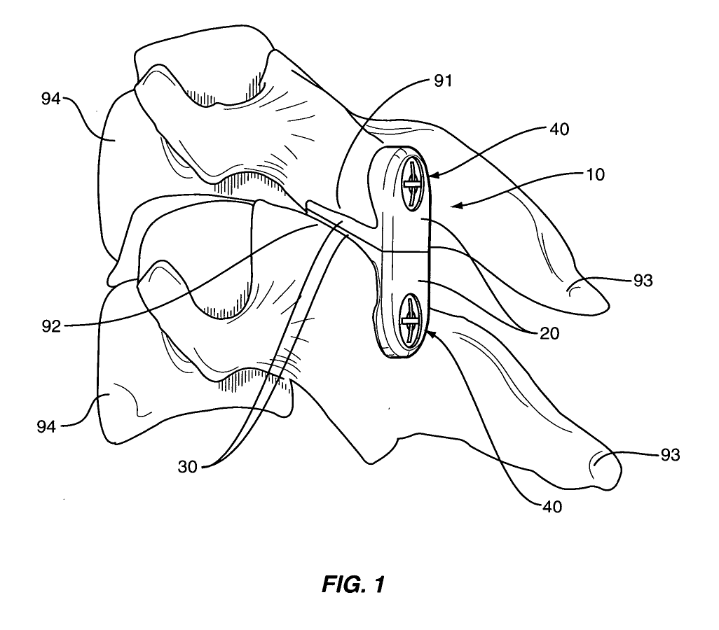

The present application is directed to a device to restore the height of a facet joint. In one embodiment illustrated in FIG. 1, the spacer 10 includes a mounting section 20 for receiving one or more mounting members 40, and an intermediate section 30. Intermediate section 30 fits within the facet joint to space apart the inferior articular process 91 and the superior articular process 92. The spacer 10 provides a cushion between the processes 91, 92 and reduces and/or eliminates bone-on-bone contact to prevent abrasion.

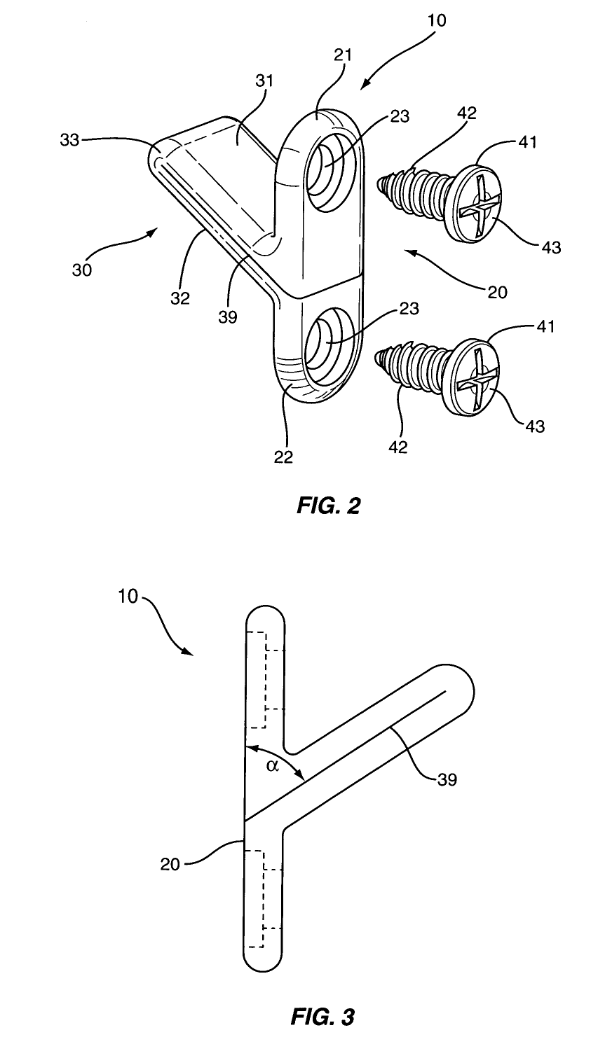

The spacer 10 is illustrated in FIG. 2 having the mounting section 20 and intermediate section 30. The spacer 10 has a folded orientation with a division 39 forming first and second sections for both the mounting section 20 and intermediate section 30. Mounting section 20 includes a first section 21 and a second section 22. The sections 21, 22 may have the same or different lengths and widths. Each section 21, 22 includes an aperture 23 sized to receive a screw 41 to mount to a vertebral member 94. Apertures 23 are sized to receive the shaft 42 and support the head 43. Although two screws 40 are illustrated to mount the spacer 10, a single screw may be adequate for a secure mount.

Intermediate section 30 includes a first section 31 and a second section 32 in an overlapping orientation forming a two-ply configuration. A fold 33 is positioned opposite from the mounting section 20. As illustrated in FIG. 3, the intermediate section is positioned at an angle a relative to the mounting section 20. In one embodiment, angle a ranges from between about 15° to about 60°, with one specific embodiment having an angle a of about 60°.

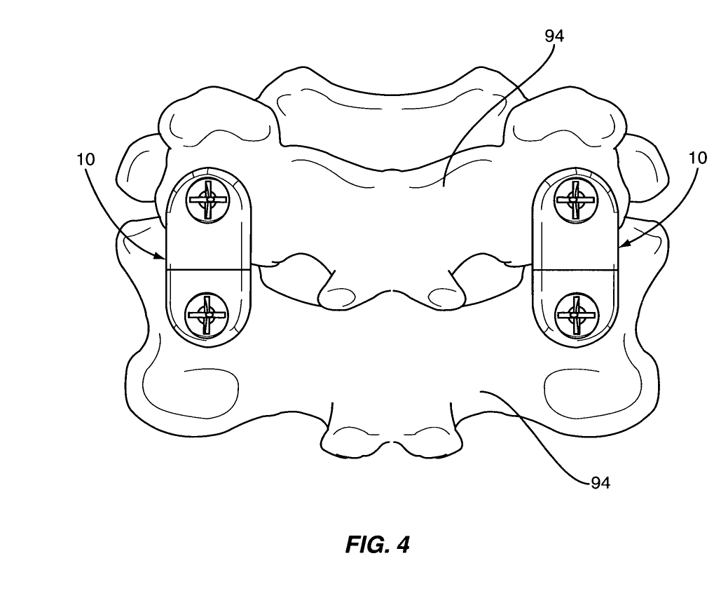

FIG. 4 illustrates a posterior view of a pair of spacers 10 mounted within the facets joints between first and second vertebral members 94. The mounting section 20 is sized to extend over the vertebral member 94 positioning the apertures 23 at positions to provide an anchor for the mounting screws 41. Intermediate section 30 is positioned within the facet joint between the inferior articular process 91 and superior articular process 92. Spacer 10 provides a cushion for the processes 91, 92 to prevent bone-on-bone contact and abrasion. This embodiment features a pair of spacers 10 mounted to the vertebral members 94 with a separate spacer 10 within each of the facet joints. The spacer 10 may also be used individually with a single spacer 10 positioned within one of the facet joints.

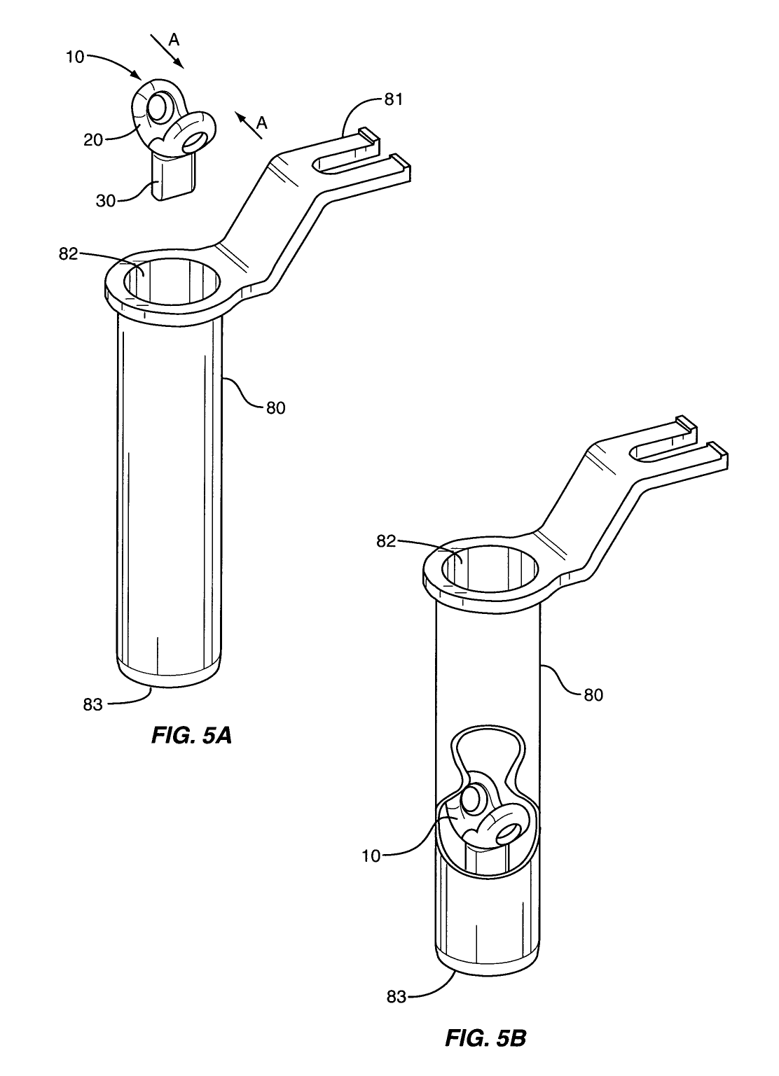

One method of insertion of the spacer 10 into the patient is illustrated in FIGS. 5A and 5B. The relatively small overall size of the spacer 10 and the flexible nature allows for a minimally invasive insertion method. Vertebral members 94 and specifically the facet joint are accessed from a posterior or lateral approach. Tube 80 is positioned for percutaneous delivery of the spacer 10 to the facet joint. Compressive force illustrated by arrows A in FIG. 5A are applied to the mounting section 20 to reduce the overall size for insertion into the tube inlet 82. Spacer 10 is then moved through the tube 80 and expelled through the outlet 83 to the facet joint. The intermediate section 30 is inserted within the facet joint between the inferior and superior articular processes 91, 92. Mounting section 20 remains on the exterior of the facet joint with apertures 23 positioned to receive mounting screws 41.

After insertion and mounting, the spacer 10 is flexible to move during movement of the vertebral members 94. A distance between the first and second sections 31, 32 may vary, such as during flexion and extension. Likewise, the spacer 10 flexes during torsional and other spinal movements.

The intermediate section 30 is sized to provide structural support between the respective inferior articular process 91 and superior articular process 92. The height is sufficient to provide support for and maintain the desired spacing between adjacent vertebral members 94 and restore the desired facet height. The spacer 10 may further prevent abrasion between the processes 91, 92 caused by bone-to-bone contact, and allow for the joint to articulate.

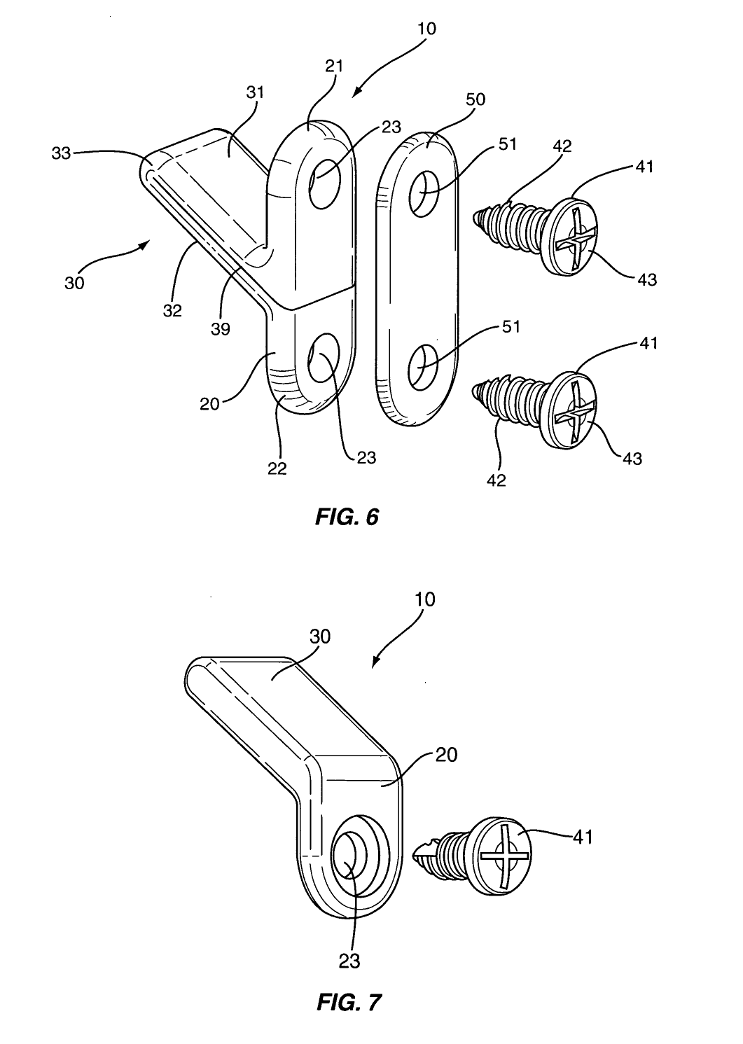

FIG. 6 illustrates a member 50 that may be mounted to the spacer 10. Member 50 provides additional tensioning support to the spacer 10 to control the facet opening and the extent of relative movement between the first and second sections 31, 32. In the event the vertebral members 94 are subjected to a force or position that would expand the facet opening beyond a predetermined amount, member 50 applies a tension force to prevent and/or reduce the amount of additional opening. Member 50 includes apertures 51 that align with the apertures 23 of the mounting section 20. Screws 41 extend through the apertures 51 to maintain the member 50. Member 50 may have the same dimensions as the mounting section 20 such as the embodiment illustrated in FIG. 6, or may have different dimensions. In one embodiment, the entire member 50 is constructed of a flexible material. In another embodiment, at least a portion of the member 50 is constructed of a flexible material. One specific embodiments includes the member 50 constructed of a stiff material around the apertures 51 and constructed of a flexible material in the intermediate area between the apertures 51.

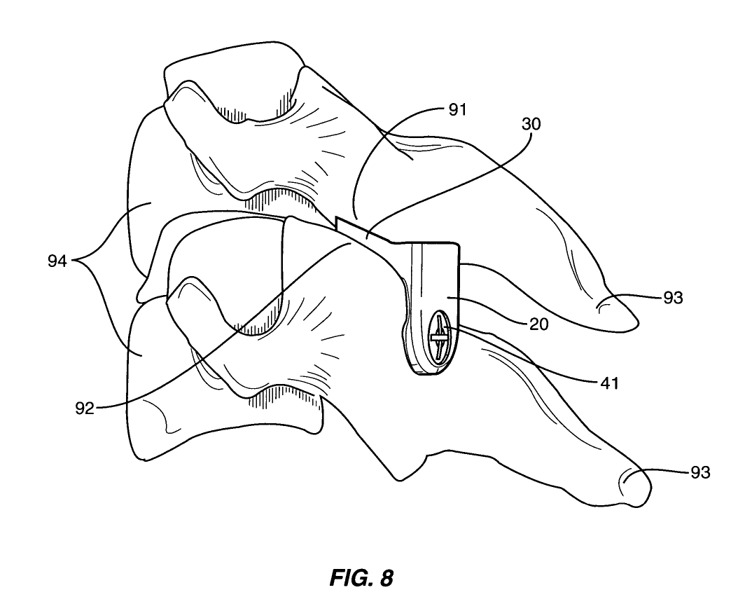

FIG. 7 illustrates another spacer embodiment having a mounting section 20 and intermediate section 30. Mounting section 20 is sized to be positioned on the exterior surface of one of the vertebral members 94 that form the facet joint. Aperture 23 is positioned to receive a screw 41 to mount the spacer 10 to the member 94. Intermediate section 30 has a thickness to fit within the facet joint and space apart the inferior and superior articular processes 91, 92. FIG. 8 illustrates this spacer embodiment connected to a vertebral member 94. A single screw 41 mounted within the inferior vertebral member 94 maintains the positioned of the spacer 10. This embodiment is used independently with no additional device mounted to the opposite vertebral member. The embodiment illustrated in FIG. 8 illustrates the spacer 10 mounted to the inferior vertebral member. This embodiment may also be constructed to instead mount to the superior vertebral member.

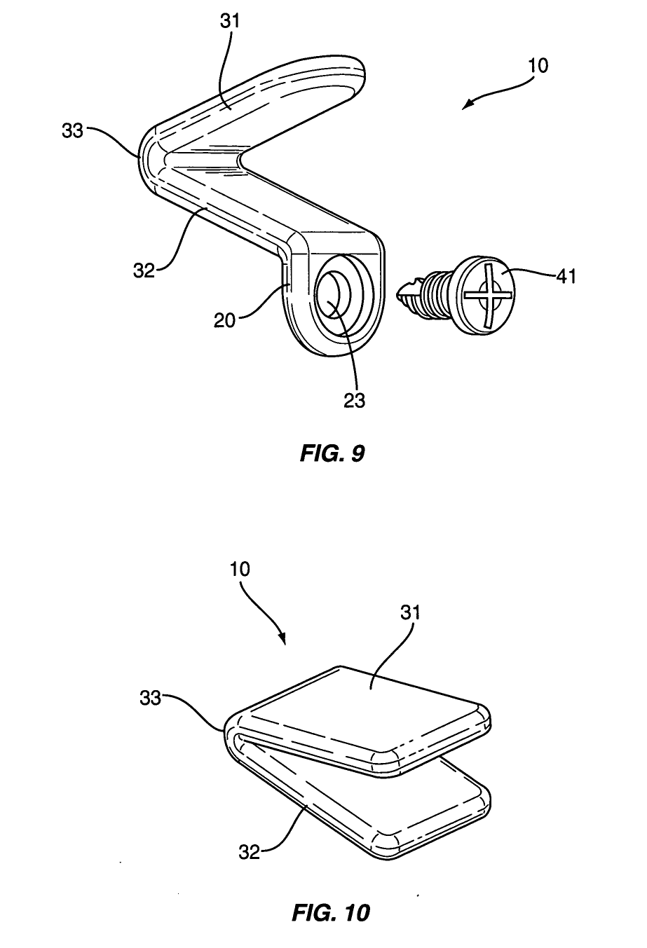

Another embodiment of a spacer 10 is illustrated in FIG. 9. A single mounting section 20 extends from one of the two members of the intermediate section 30. Specifically, the mounting section 20 is positioned at an end of the second member 32. First member 31 is maintained within the facet joint through the connection at the fold 33 to the second member 32. The first member 31 extends a distance outward beyond the fold 33 in the general direction of the mounting section 20. In one embodiment, the first member 31 extends outward from the fold 33 for an end to be positioned short of the mounting section 20 (i.e., the end is positioned between the fold 33 and the mounting section 20).

FIG. 10 illustrates another embodiment having an intermediate section comprising first and second sections 31, 32 connected at a fold 33. This embodiment does not include a mounting section. The first and second sections 31, 32 may have the same or different lengths. Therefore, the entirety of the spacer 10 may have a two-ply configuration, or may have a section having two-ply and a section having a single-ply configuration.

A variety of different mounting means may be used for connecting the spacer 10 within the facet joint. In several illustrated embodiments, one or more screws 41 mount the spacer 10. Other mechanical fasteners may be used for mounting the spacer 10, such as a staple. Adhesives may also be used for mounting the spacer 10. Examples of adhesives include a two-part epoxy and UV-curing epoxy. The adhesives may be applied to the mounting section 20, or to the intermediate section 30. By way of example, the spacer embodiment illustrated in FIG. 10 may be mounted with an adhesive mounted on the surface of one or both of the first and second section 31, 32. In another embodiment, the spacer 10 is maintained in position by contact between the inferior and superior articular processes 91, 92 without mechanical fasteners or adhesives.

In embodiments having a two-ply intermediate section 30, the angular positioned of the first and second sections 31, 32 may vary depending upon the application. The amount of angle and the construction of the spacer 10 results in the spacer 10 applying various outward forces to the facet joint. Further, the angular position may vary depending upon the movement of the vertebral members 94. In one orientation, the angular positioning may be more or less than in a second orientation.

Spacer 10 may be constructed from a flexible, bio-compatible material material, such as an elastomer or flexible composite material, to name a few. Suitable elastomers include silicone, polyurethane, copolymers of silicone and polyurethane, hydrogels, polyolefins, such as polyisobutylene and polyisoprene, neoprene, nitrile, vulcanized rubber and combinations thereof. In another embodiment, spacer 10 is an inflatable sheath constructed to receive an injectable elastic material. The sheath is constructed from a flexible, bio-compatible material as described above. The sheath has a reduced size in a first orientation, and then expands to a working size upon insertion of the elastic material. The injectable elastic material may include silicone, polyurethane, copolymers of silicone and polyurethane, hydrogels and combinations thereof. The flexible sheath may also be filled with saline. The injectable elastic material may also include collagen with the application of an elastic semipermeable membrane for the sheath. Member 50 may be constructed from a flexible, bio-compatible material, such as an elastomer, or flexible composite material as described above.

The embodiment of FIGS. 5A and 5B illustrate the mounting section 20 being compressed to fit within the tube 80. Spacer 10 may also be sized to fit within the tube 80 without requiring compression or other size-reducing measures.

An amount of space and angular orientation may vary between the first and second sections 31, 32 of the intermediate section 30. In one embodiment as illustrated in FIG. 2, the inner faces of the first and second sections 31, 32 are in contact with each other. In another embodiment as illustrated in FIG. 10, the sections 31, 32 are spaced apart forming a pre-loaded angle. The device assumes the pre-loaded angle when no external forces are applied. Once inserted within the facet joint, the angle will vary during movement of the joint. By way of example, during insertion the sections 31, 32 may be compressed and the faces in contact to reduce the overall size of the device. Once mounted within the facet joint, the sections 31, 32 may separate apart. Further, the space and angular orientation may vary during movement of the patient after insertion of the device 10. During a first posture, the sections 31, 32 may be in contact, and in a second posture a space may exist between the sections 31, 32.

Spacer 10 can be inserted using a minimally invasive procedure to augment the bearing surface and/or restore the height of the facet joint. The spacer 10 is not as disruptive to the facet joint as a total facet joint replacement. Further, the spacer 10 is not nearly as technically demanding and may not be a final stage procedure as a total facet joint replacement.

The term “distal” is generally defined as in the direction of the patient, or away from a user of a device. Conversely, “proximal” generally means away from the patient, or toward the user. Spatially relative terms such as “under”, “below”, “lower”, “over”, “upper”, “superior”, “inferior”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting.

The present invention may be carried out in other specific ways than those herein set forth without departing from the scope and essential characteristics of the invention. In one embodiment, spacer 10 is constructed of a rigid material. In one embodiment, member 50 is constructed of a rigid material. The present embodiments are, therefore, to be considered in all respects as illustrative and not restrictive, and all changes coming within the meaning and equivalency range of the appended claims are intended to be embraced therein.

What is claimed is:

1. A device to space a facet joint formed between first and second vertebral members, the device comprising:
a mounting section sized to extend over a face of the first vertebral members;
a first intermediate section integral with and extending outward from the mounting section;
a second intermediate section integral with the first intermediate section and forming a two layer construction sized to fit within the facet joint between the first and second vertebral members; and
a fold connecting the first and second intermediate sections;
the device being constructed of a flexible material that permits movement of the facet joint.

2. The device of claim 1, wherein the fold is positioned opposite from the mounting section.

3. The device of claim 2, wherein an end of the second intermediate section extends outward and is positioned between the fold and the mounting section.

4. The device of claim 1, further comprising a second mounting section integral with the second intermediate section and sized to extend over a second vertebral member face.

5. The device of claim 4, wherein the mounting section and the second mounting section are substantially aligned within a common plane.

6. The device of claim 4, wherein a length of the mounting section and the first intermediate section is the same as the second mounting section and the second intermediate section.

7. The device of claim 4, further comprising a flexible tension member sized to extend across sections of the mounting section and the second mounting section to control an extent of relative movement between the first and second intermediate members.

8. The device of claim 7, wherein the tension member is attached to the mounting section at a first mounting section aperture and to the second mounting section at a second mounting section aperture.

9. The device of claim 1, wherein the mounting section comprises an aperture to receive a fastening device to mount the mounting section to one of the first and second vertebral members.

10. The device of claim 1, wherein an angle formed between the first and second intermediate sections changes during movement of the facet joint.

11. A device to space a facet joint formed between first and second vertebral members, the device comprising:
a two-ply intermediate section sized to fit within the facet joint; and
a mounting section sized to extend over a portion of one of the first and second vertebral members;
the intermediate section and the mounting section constructed from a single member and being flexible to permit movement of the facet joint.

12. The device of claim 11, wherein the intermediate section comprises a first section folded onto a second section.

13. The device of claim 12, wherein the first section has a greater length than the second section.

14. The device of claim 12, wherein a fold is positioned opposite from the mounting section.

15. The device of claim 11, further comprising a second mounting section extending from the intermediate section and extending away from the mounting section.

16. The device of claim 15, further comprising a tension member connected to an exterior face of the mounting section and the second mounting section, the tension member constructed of a flexible material to exert a force when loaded on the mounting sections to prevent movement relative outward movement between the mounting sections.

17. The device of claim 11, wherein the intermediate section includes a first ply that contacts the first vertebral member and a second ply that contacts the second vertebral member.

18. A device to space a facet joint formed between first and second vertebral members, the device comprising:
a first section having a width to fit within the facet joint with a first face contacting the first vertebral member and a second face contacting the second vertebral member; and
a second section extending at an angle from the first section and extending over at least a portion of an exterior face of one of the first and second vertebral members.

19. The device of claim 18, wherein the first section and the second sections are a unitary member and constructed of a flexible material to allow for relative movement between the first and second sections during movement of the facet joint.

20. A device to space a facet joint formed between first and second vertebral members, the device comprising:
a first intermediate section; and
a second intermediate section positioned to extend over at least a portion of the first intermediate section;
the intermediate sections having a folded orientation and being constructed from a single flexible member that provides for movement of the facet joint.

21. The device of claim 20, wherein an exterior face of the first and second intermediate sections is substantially flat.

22. The device of claim 20, wherein the first and second intermediate sections have an equal length.

23. The device of claim 20, wherein the first and second intermediate sections are constructed of a single elongated member.

24. The device of claim 20, further comprising a mounting section extending from an end of one of the first and second intermediate sections, the mounting section positioned to extend over a face of one of the vertebral members.

25. The device of claim 20, wherein the first and second intermediate sections are spaced apart at a preloaded angle prior to insertion into the facet joint.

26. A device to space a facet joint formed between first and second vertebral members, the device comprising:
a first intermediate section having an outer surface for positioning against the first vertebral member and an inner surface facing away from the first vertebral member; and
a second intermediate section having an outer surface for positioning against the second vertebral member and an inner surface facing away from the second vertebral member;
the intermediate sections being formed from a single member and having an overlapping orientation with the inner surfaces facing one another.

27. The device of claim 26, further comprising a mounting section extending from an end of one of the first and second intermediate sections, the mounting section positioned to extend over a face of one of the vertebral members.

28. The device of claim 26, wherein the single member is constructed of a flexible material to allow movement of the facet joint.

29. A device to space a facet joint formed between first and second vertebral members, the device comprising:
a first mounting section sized to extend over a section of the first vertebral member;
a second mounting section sized to extend over a section of the second vertebral member;
an intermediate section connected to each of the first and second mounting sections, the intermediate section having first and second members in an overlapping configuration sized to fit between the first and second vertebral members; and
a flexible outer member mounted to the first and second mounting sections to apply tension when loaded to the first and second mounting sections.

30. The device of claim 29, wherein the member is mounted to an opposite side of the first and second mounting sections from the intermediate members.

31. The device of claim 29, wherein the first and second mounting sections are connected together along a fold.

32. The device of claim 31, wherein the first and second mounting sections and the intermediate section are constructed from a single element.

33. The device of claim 29, wherein the first and second mounting sections are substantially aligned within a common plane.

34. The device of claim 29, wherein apertures on the outer member align with corresponding apertures in the first and second mounting sections.

35. A method of spacing a facet joint formed between first and second vertebral members, the method comprising the steps of:
positioning a unitary, two layer intermediate section within the facet joint and spacing apart the first and second vertebral members;
attaching a first mounting section that extends outward from a first layer of the intermediate section to the first vertebral member; and
attaching a second mounting section that extends outward from a second layer of the intermediate section to the second vertebral member.

36. The method of claim 35, further comprising applying tension to the first and second mounting sections by attaching a member to outer faces of the first and second mounting sections.

37. The method of claim 35, further comprising positioning a fold section of the intermediate section within the facet joint.

38. The method of claim 35, further comprising changing an angle between first and second members of the intermediate section during movement of the facet joint.
